(19) United States
(12) Patent Application Publication
(10) Pub. No.: US 2001/0055599 A1
(43) Pub. Date: Dec. 27, 2001

(54) COMPOSITIONS CONTAINING SELECT LIQUID POLYOL FATTY ACID POLYESTERS

(76) Inventors: PAUL JOSEPH DRZEWIECKI, MAINEVILLE, OH (US); JOSEPH ANTHONY LISTRO, LOVELAND, OH (US)

Correspondence Address: THE PROCTER & GAMBLE COMPANY, PATENT DIVISION, SHARON WOODS TECHINICAL CENTER, 11511 REED HARTMAN HIGHWAY, CINCINNATI, OH, 45241, US

(21) Appl. No.: 08/862,344

(22) Filed: May 23, 1997

(51) Int. Cl. A61K 6/00; A61K 7/00

(52) U.S. Cl. 424/401

(57) ABSTRACT

Disclosed are compositions suitable for topical application to human hair or skin, which comprise a combination of a liquid polyol fatty acid polyester having a melting point of from about −30° C. to about 30° C., and a solid oil other than a solid polyol fatty acid polyester having a melting point of above about 30° C. to about 250° C., wherein said liquid polyol fatty acid polyester has a polyol moiety and at least 1 fatty acid moiety, the polyol moiety having at least 4 free hydroxyl groups wherein at least 60% of these free hydroxyl groups are esterified with one or more fatty acid moieties having from about 8 to about 22 carbon atoms. The composition preferably further comprises a topical carrier for the liquid polyol fatty acid polyester and solid oil combination. The composition provides effective emolliency and aesthetic benefits.

### TECHNICAL FIELD

The present invention relates to compositions suitable for topical application to human hair or skin, which comprise a select combination of liquid polyol fatty acid polyesters having a melting point of from about −30° C. to about 30° C. and solid oils having a melting point above about 30° C.

### BACKGROUND OF THE INVENTION

Topical compositions containing emollients have been used for many years in the treatment of human hair or skin. For example, occlusive hydrocarbons such as petrolatum have been used as topical emollients to provide a protective film onto human skin to prevent water loss to the environment. Petrolatum has also been used in hair care products such as conditioners and grooming aids.

However, the most effective and widely used compositions containing occlusive emollients suffer from negative aesthetic qualities such as greasiness and stickiness. Moreover, some occlusive emollients that are used to provide a protective film on the skin results in clogging the skin pores and preventing the flow of oxygen. This obstruction of the skin surface or blockage of the passage or circulation of air and moisture limits the use of such heavy, occlusive emollient materials. Additionally, European Patent No. 458,600 B1, published Mar. 2, 1994, discloses occlusive skin care compositions containing a polyol fatty acid polyester having at least 4 free hydroxyl groups, at least 60% of which are esterified with one or more fatty acids having from 8 to 22 carbon atoms which can form an occlusive film on the skin following topical application thereon. U.S. Pat. No. 5,160,738, to Macaulay et al., issued Nov. 3, 1992, further discloses occlusive compositions containing a blend of two or more polyol fatty acid polyesters which has the appearance and physical properties of petrolatum. However, these compositions also have the disadvantage of being heavy and can clog the skin pores preventing the flow of oxygen.

It has now been found that compositions containing occlusive emollients can be formulated without being heavy, sticky, or greasy. These compositions contain a select combination of liquid polyol fatty acid polyesters having a melting point of from about −30° C. to about 30° C., and solid oils having a melting point above about 30° C., and such compositions are applicable in a variety of products to provide both efficacy and aesthetic benefits.

It is therefore an object of the present invention to provide a composition which has both effective emolliency and acceptable aesthetic qualities, and which contain an occlusive emollient used in combination with a solid oil. It is a further object of the present invention to provide topical compositions containing polyol fatty acid polyesters which are not heavy, sticky, and greasy, and which are effective in the treatment of human hair or skin.

### SUMMARY OF THE INVENTION

The present invention relates to a composition suitable for topical application to the human hair or skin, which comprises a combination of a liquid polyol fatty acid polyester having a melting point of from about −30° C. to about 30° C., and a solid oil other than a solid polyol fatty acid polyester having a melting point of above about 30° C. to about 250° C., wherein said liquid polyol fatty acid polyester has a polyol moiety and at least 1 fatty acid moiety, the polyol moiety having at least 4 free hydroxyl groups wherein at least 60% of these free hydroxyl groups are esterified with one or more fatty acid moieties having from about 8 to about 22 carbon atoms. The composition preferably further comprises a topical carrier for the liquid polyol fatty acid polyester and solid oil combination.

All percentages and ratios used herein are by weight and all measurements made are at 25° C., unless otherwise designated. The invention hereof can comprise, consist of, or consist essentially of, the essential as well as optional ingredients and components described herein.

### DETAILED DESCRIPTION OF THE INVENTION

The term “topical composition” as used herein means a composition suitable for topical application to human hair or skin. The term is used to encompass a wide variety of personal care, beauty care, and cosmetic compositions. Nonlimiting examples of topical compositions include lotions, creams, hand and body lotions, skin conditioning lotions and creams, skin protectant compositions, sunscreen compositions, cold creams, anti-acne compositions, skin renewal products, non-lathering cleansing lotions, moisturizers, facial moisturizers, make-ups, foundations, lipsticks, lip protectants, skin cleansers, hand, face, and body cleansers, shower products, shampoos, and the like.

The term “topical carrier”, as used herein, is well-known to one of ordinary skill in the art, and means one or more compatible solid or liquid filler diluents or vehicles which are suitable for administration to a human. The term “compatible”, as used herein, means that the components of the topical carrier are capable of being comingled with the components of the present invention, and with each other, in a manner such that there is no interaction which would substantially reduce the efficacy or aesthetics of the cosmetic composition under ordinary use situations. The topical carrier must be a pharmaceutically acceptable carrier. The term “pharmaceutically-acceptable”, as used herein, means that the topical carrier must be of sufficiently high purity and be suitable for use in contact with human hair or skin without undue toxicity, incompatibility, instability, allergic response, and the like.

The melting point of the liquid polyol fatty acid polyesters and solid oils can be determined using conventional techniques. Such techniques are well defined in the art, and include thermometric as well as calorimetry methodology. A particularly preferred technique for determining the melting point is described in U.S. Pat. No. 5,306,514, to Letton et al., issued Apr. 26, 1994, which is incorporated by reference herein in its entirety. This technique typically involves measuring melting points using Differential Scanning Calorimetry (DSC) whereby a scanning temperature of 5° C./minute is used to measure the melting point. The melting point is the temperature at the intersection of the baseline, i.e. the specific heat line, with the line tangent to the trailing edge of the endothermic peak.

### Liquid Polyol Fatty Acid Polyester

The composition of the present invention comprises a liquid polyol fatty acid polyester at concentrations ranging from about 0.1% to about 99.9%, preferably from about 0.5% to about 75%, more preferably from about 1% to about 50%, even more preferably from about 2% to about 25%, by weight of the composition. These liquid polyol fatty acid polyesters have melting points below about 30° C., and are derived from any aliphatic or aromatic polyol which has at least 4 free hydroxyl groups, of which at least 60% of these free hydroxyl groups are then esterified with one or more fatty acids having from about 8 to about 22 carbon atoms. The fatty acids can also be described as carboxylic acids, because the terms fatty acid and carboxylic acid are often used interchangeably by those skilled in the art.

The liquid polyol polyesters employed in this invention comprise certain polyols, especially sugars or sugar alcohols, esterified with one or more fatty acid groups. Accordingly, the polyol starting material must have at least four esterifiable hydroxyl groups. Examples of preferred polyols are sugars, including monosaccharides and disaccharides, and sugar alcohols. Examples of monosaccharides containing four hydroxyl groups are xylose and arabinose and the sugar alcohol derived from xylose, which has five hydroxyl groups, i.e., xylitol. The monosaccharide, erythrose, is not suitable in the practice of this invention since it only contains three hydroxyl groups, but the sugar alcohol derived from erythrose, i.e., erythritol, contains four hydroxyl groups and accordingly can be used. Suitable five hydroxyl group-containing monosaccharides are galactose, fructose, and sorbose. Sugar alcohols containing six hydroxyl groups derived from the hydrolysis products of sucrose, as well as glucose and sorbose, e.g., sorbitol, are also suitable. Examples of disaccharide polyols which can be used include maltose, lactose, and sucrose, all of which contain eight hydroxyl groups.

The polyols used in the liquid polyol esters of the present invention have from about 4 to about 12, more preferably from about 4 to about 11, and most preferably from about 4 to about 8 hydroxyl groups. Preferred polyols for preparing the polyesters for use in the present invention are selected from the group consisting of erythritol, xylitol, sorbitol, glucose, and sucrose. Sucrose is especially preferred.

The preferred polyol starting material having at least four hydroxyl groups must be esterified on at least 60% of the hydroxyl groups with a fatty acid containing from about 8 to about 22 carbon atoms, preferably from about 8 to about 18 carbon atoms. Examples of such fatty acids include caprylic, capric, lauric, myristic, myristoleic, palmitic, palmitoleic, stearic, oleic, ricinoleic, linoleic, linolenic, eleostearic, arachidic, arachidonic, behenic, and erucic acids. The fatty acids can be derived from naturally occurring or synthetic fatty acids; they can be saturated or unsaturated, including positional and geometrical isomers. However, in order to provide liquid polyesters of the type used herein, at least about half of the fatty acid incorporated into the polyester molecule must be unsaturated. Oleic and linoleic acids, and mixtures thereof, are especially preferred.

The liquid polyol fatty acid polyesters useful in this invention must contain one or more fatty acid ester groups. It is not necessary that all of the hydroxyl groups of the polyol be esterified with fatty acids, but it is preferable that at least 60% of the hydroxyl groups are esterified with fatty acid ester groups. Most preferably, substantially all of the hydroxyl groups of the polyol are esterified with fatty acids, i.e., the polyol moiety is substantially completely esterified. The fatty acids esterified to the polyol molecule can be the same or mixed, but as noted above, a substantial amount of the unsaturated acid ester groups must be present to provide liquidity.

To illustrate the above points, a sucrose di-fatty acid ester would be suitable for use herein, but is not preferred because it has more than two unesterified hydroxyl groups. Likewise, a sucrose tetra-fatty acid ester would be suitable, but is not preferred because it also has more than two unesterified hydroxyl groups. Highly preferred compounds in which all the hydroxyl groups are esterified with fatty acids include the liquid sucrose octa-substituted fatty acid esters.

The following are non-limiting examples of specific liquid polyol fatty acid polyesters containing one or more fatty acid ester groups suitable for use in the present invention: glucose oleate, the glucose ester of soybean oil fatty acids (unsaturated), the mannose ester of mixed soybean oil fatty acids, the galactose ester of oleic acid, the arabinose ester of linoleic acid, xylose linoleate, sorbitol oleate, sucrose oleate, glucose dioleate, the glucose diesters of soybean oil fatty acids (unsaturated), the mannose diesters of mixed soybean oil fatty acids, the galactose diesters of oleic acid, the arabinose diesters of linoleic acid, xylose dilinoleate, sorbitol dioleate, sucrose dioleate, glucose trioleate, the glucose triesters of soybean oil fatty acids (unsaturated), the mannose triesters of mixed soybean oil fatty acids, the galactose triesters of oleic acid, the arabinose triesters of linoleic acid, xylose trilinoleate, sorbitol trioleate, sucrose trioleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, sorbitol tetraoleate, galactose pentaoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Preferred are liquid polyol esters selected from the group consisting of sucrose pentaoleate, sucrose hexaoleate, sucrose heptaoleate, sucrose octaoleate, and mixtures thereof. More preferred are sucrose hexaoleate, sucrose heptaoleate, sucrose octaoleate, and mixtures thereof.

The preferred liquid polyol fatty acid polyesters of the present invention have melting points of from about −30° C. to about 30° C., preferably from about −30° C. to about 27.5° C., and more preferably from about −30° C. to about 25° C. The melting points are measured using conventional techniques.

The polyol fatty acid polyesters suitable for use herein can be prepared by a variety of methods well known to those skilled in the art. These methods include: transesterification of the polyol with methyl, ethyl or glycerol fatty acid esters using a variety of catalysts; acylation of the polyol with a fatty acid chloride; acylation of the polyol with a fatty acid anhydride; and acylation of the polyol with a fatty acid, per se. See U.S. Pat. No. 2,831,854;, U.S. Pat. No. 4,005,196, to Jandacek, issued Jan. 25, 1977; and U.S. Pat. No. 4,005,196, to Jandacek, issued Jan. 25, 1977, all of which are incorporated by reference herein in their entirety.

### Solid Oil

The composition of the present invention comprises a solid oil used in combination with the liquid polyol fatty acid polyester described herein. The solid oil can be used as an individual solid oil or a combination of solid oils, and is included at concentrations ranging from about 0.1% to about 99.9%, preferably from about 0.5% to about 75%, more preferably from about 1% to about 50%, even more preferably from about 2% to about 25%, by weight of the composition.

As used herein the term “solid oils” refers to those materials which have a melting point above about 30° C., preferably above about 30° C. to about 250° C., more preferably from about 37° C. to about 100° C., even more preferably from about 37° C. to about 80° C. Examples of suitable solid oils include, but are not limited to, petrolatum, highly branched hydrocarbons, fatty alcohols, fatty acid esters, vegetable oils, hydrogenated vegetable oils, polypropylene glycols, alpha-hydroxy fatty acids, fatty acids having from about 10 to about 40 carbon atoms, alkyl amides of di and/or tri-basic carboxylic acids, n-acyl amino acid derivatives, and mixtures thereof. Solid oils useful in the cosmetic composition of the present invention are further described in U.S. Pat. No. 4,919,934, to Deckner et al., issued Apr. 24, 1990, which is incorporated herein by reference in its entirety.

Suitable highly branched hydrocarbons for use herein include hydrocarbon compounds having from about 17 to about 40 carbon atoms. Nonlimiting examples of these hydrocarbon compounds include squalane, cholesterol, lanolin, docosane (i.e. a C₂₂ hydrocarbon), and isoparaffins.

Suitable fatty alcohols for use herein include monohydric alcohols, ethoxylated fatty alcohols, and fatty alcohol esters, excluding the ethoxylated fatty alcohols and fatty alcohol esters useful as emulsifiers herein. Specific examples of commercially available fatty alcohols include, but are not limited to, Unilin 550, Unilin 700, Unilin 425, Unilin 400, Unilin 350, and Unilin 325, all supplied by Petrolite. Suitable ethoxylated fatty alcohols include, but are not limited, Unithox 325, Unithox 400, and Unithox 450, Unithox 480, Unithox 520, Unithox 550, Unithox 720, Unithox 750, all of which are available from Petrolite. Non-limiting examples of suitable esters of fatty alcohols include tri-isostearyl citrate, ethyleneglycol di-12-hydroxystearate, tristearylcitrate, stearyl octanoate, stearyl heptanoate, trilaurylcitrate.

Suitable fatty acid esters for use herein include ester waxes, monoglycerides, diglycerides, triglycerides and mixtures thereof. Non-limiting examples of suitable ester waxes include stearyl stearate, stearyl behenate, palmityl stearate, stearyl octyldodecanol, cetyl esters, cetearyl behenate, behenyl behenate, ethylene glycol distearate, ethylene glycol dipalmitate, and beeswax. Examples of commercial ester waxes include Kester waxes from Koster Keunen, Crodamol SS from Croda and Demalcare SPS from Rhone Poulenc.

Vegetable oils and hydrogenated vegetable oils which are solid or semi-solid at ambient temperatures of from about 20° C. to about 25° C. are also useful herein. Examples of suitable vegetable oils and hydrogenated vegetable oils include butterfat, chicken fat, goose fat, horse fat, lard (fatty tissue) oil, rabbit fat, sardine oil, tallow (beef), tallow (mutton), chinese vegetable tallow, babassu oil, cocoa butter, coconut oil, palm oil, palm kernal oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, derivatives thereof and mixtures thereof.

Suitable polypropylene glycols for use herein include C₄-C₁₆ alkyl ethers of polypropylene glycols, and C₁-C₁₆ carboxylic acid esters of polypropylene glycols. Nonlimiting examples of these materials include PPG-14 butyl ether, PPG-15 stearyl ether, PPG-9, PPG-12, PPG-15, PPG-17, PPG-20, PPG-26, PPG-30, PPG-34, and mixtures thereof.

Examples of suitable alpha-hydroxy fatty acids and fatty acids having from about 10 to about 40 carbon atoms include 12-hydroxystearic acid, 12-hydroxylauric acid, 16-hydroxyhexadecanoic acid, behenic acid, eurcic acid, stearic acid, caprylic acid, lauric acid, isostearic acid, and mixtures thereof. Examples of some suitable fatty acids are further described in U.S. Pat. No. 5,429,816, issued to Hofrichter et al. on Jul. 4, 1995; and U.S. Pat. No. 5,552,136, issued to Motley on Sep. 3, 1996, which descriptions are incorporated herein by reference.

Suitable alkyl amides of di and/or tri-basic carboxylic acids for use herein include disubstituted or branched monoamides, monosubstituted or branched diamides, triamides, and mixtures thereof. Some specific examples of alkyl amides of di- and tri-basic carboxylic acids include, but are not limited to, alkyl amides of citric acid, tricarballylic acid, aconitic acid, nitrilotriacetic acid and itaconic acid such as 1,2,3-propane tributylamide, 2-hydroxy-1,2,3-propane tributylamide, 1-propene-1,2,3-trioctylamide, N,N′,N″-tri(methyldecylamide)amine, 2 docecyl-N,N′-dibutylsuccinamide, and mixtures thereof. Other suitable amides include the n-acyl amino acid derivatives described in U.S. Pat. No. 5,429,816, issued to Hofrichter et al. on Jul. 4, 1995.

### Topical Carrier

The composition of the present invention comprises from about 0.1% to about 99.9%, preferably from about 50% to about 99%, and more preferably from about 60% to about 95% by weight of a topical carrier for the liquid polyol fatty acid polyester and solid oil combination, and for any other optional components of the present invention.

The liquid polyol fatty acid polyester and solid oil combination of the present invention can be formulated into a wide variety of product types, including creams, lotions, milks, gels, hand and body lotions, cold creams, non-lathering cleansing lotions, facial moisturizers, sunscreens, anti-acne preparations, topical analgesics, mascaras, lipsticks, skin cleansers, hand, face, and body cleansers, shower products, shampoos, and the like. The carriers and any additional components required to formulate such products vary with product type and can be routinely chosen by one skilled in the art.

The topical carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; U.S. Pat. No. 4,254,105 to Fukuda et al., issued Mar. 3, 1981; U.S. Pat. No. 4,976,953, to Orr et al., issued Dec. 11, 1990; U.S. Pat. No. 5,073,372, to Turner et al., issued Dec. 17, 1991; U.S. Pat. No. 5,585,104, to Ha et al., issued Dec. 17, 1996; U.S. Pat. No. 5,607,678, to Moore et al., issued Mar. 4, 1997; U.S. Pat. No. 5,607,980, to McAtee et al., issued Mar. 4, 1997; and U.S. Pat. No. 5,618,522, to Kaleta et al., issued Apr. 8, 1997.

The topical carrier can also comprise an oil-in-water emulsion system having complex structures such as liquid crystals and crystalline gel networks. The nature of liquid crystals, the formation of liquid crystals, the properties and advantages of liquid crystals are described further in G. Dahms, Properties of O/W Emulsions With Anisotropic Lameliar Phases, 101 Cosmetics & Toiletries, 113-115 (1986); P. Loll, Liquid Crystals in Cosmetic Emulsions, ICI Surfactants' Publication RP94-93E; and G. M. Eccleston, Multiple-Phase Oil-In-Water Emulsions, 41, J. Soc. Cosmet. Chem., 1-22, (January/February 1990); all of which are incorporated herein by reference in their entirety.

### Additional Components

A wide variety of additional components can be employed in the compositions herein. Non-limiting examples include the following:

Pharmaceutical Actives

The compositions of the present invention can comprise a safe and effective amount of a pharmaceutical active. The phrase “safe and effective amount”, as used herein, means an amount of an active high enough to significantly or positively modify the condition to be treated, but low enough to avoid serious side effects (at a reasonable benefit/risk ratio), within the scope of sound medical judgment. A safe and effective amount of the pharmaceutical active will vary with the specific active, the ability of the composition to penetrate the active through the skin, the amount of composition to be applied, the particular condition being treated, the age and physical condition of the patient being treated, the severity of the condition, the duration of the treatment, the nature of concurrent therapy, and like factors.

The pharmaceutical actives which can be used in the compositions of the present invention preferably comprise from about 0.1% to about 20% by weight of the compositions, more preferably from about 0.1% to about 10%, and most preferably from about 0.1% to about 5%. Mixtures of pharmaceutical actives may also be used.

Nonlimiting examples of pharmaceutical actives can include the following:

Useful pharmaceutical actives in the compositions of the present invention include anti-acne drugs. Anti-acne drugs for use herein include the keratolytics such as salicylic acid, sulfur, lactic acid, glycolic, pyruvic acid, resorcinol, and N-acetylcysteine; retinoids such as retinoic acid and its derivatives (e.g., cis and trans); antibiotics and antimicrobials such as benzoyl peroxide, octopirox, erythromycin, zinc, tetracyclin, triclosan, azelaic acid and its derivatives, phenoxy ethanol and phenoxy propanol, ethylacetate, clindamycin and meclocycline; sebostats such as flavinoids; alpha and beta hydroxy acids; and bile salts such as scymnol sulfate and its derivatives, deoxycholate, and cholate. Preferred anti-acne actives are those selected from the group consisting of salicylic acid, sulfur, resorcinol, lactic acid, zinc, erythromycin, benzoyl peroxide, and mixtures thereof. More preferred is salicylic acid.

Useful pharmaceutical actives in the compositions of the present invention include non-steroidal anti-inflammatory drugs (NSAIDS). The NSAIDS can be selected from the following categories: propionic acid derivatives; acetic acid derivatives; fenamic acid derivatives; biphenylcarboxylic acid derivatives; and oxicams. All of these NSAIDS are fully described in the U.S. Pat. No. 4,985,459 to Sunshine et al., issued Jan. 15, 1991, incorporated by reference herein. Most preferred are the propionic NSAIDS including but not limited to aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Also useful are the steroidal anti-inflammatory drugs including hydrocortisone and the like.

Useful pharmaceutical actives in the compositions of the present invention include antipruritic drugs. Antipruritic drugs preferred for inclusion herein include pharmaceutically-acceptable salts of methdilizine and trimeprazine.

Useful pharmaceutical actives in the compositions of the present invention include anesthetic drugs. Anesthetic drugs preferred for inclusion herein include pharmaceutically-acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Useful pharmaceutical actives in the compositions of the present invention include antimicrobial drugs (antibacterial, antifungal, antiprotozoal and antiviral drugs). Antimicrobial drugs preferred for inclusion herein include pharmaceutically-acceptable salts of b-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, paromomycin, streptomycin, tobramycin, miconazole and amanfadine. Antimicrobial drugs preferred for inclusion herein include tetracycline hydrochloride, erythromycin estolate, erythromycin stearate (salt), amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole.

Also useful herein are sunscreening agents. A wide variety of sunscreening agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Preferred among those sunscreens which are useful in the compositions of the instant invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof.

Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991; these two references are incorporated by reference herein in their entirety. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. These sunscreening agents provide higher efficacy, broader UV absorption, lower skin penetration and longer lasting efficacy relative to conventional sunscreens. Especially preferred examples of these sunscreens include those selected from the group consisting of 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof.

Generally, the compositions can comprise from about 0.5% to about 20% of the sunscreens useful herein. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978, which is incorporated herein by reference in its entirety.

Also useful in the compositions of the present invention are sunless tanning agents including dihydroxyacetone, glyceraldehyde, indoles and their derivatives, and the like. These sunless tanning agents can also be used in combination with the sunscreen agents.

Other useful actives include skin bleaching (or lightening) agents including but not limited to hydroquinone, ascorbic acid, kojic acid and sodium metabisulfite.

Humectants and Moisturizers

The compositions of the present invention can also contain one or more additional humectant or moisturizing materials other than those described herein. A variety of these materials can be employed and each can be present at a level of from about 0.1% to about 20%, more preferably from about 1% to about 10% and most preferably from about 2% to about 5%, by weight of the composition. These materials include guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; sugars and starches; sugar and starch derivatives (e.g., alkoxylated glucose); hyaluronic acid; lactamide monoethanolamine; acetamide monoethanolamine; and mixtures thereof.

Emulsifiers

The compositions herein can contain various emulsifiers. These emulsifiers are useful for emulsifying the various carrier components of the compositions herein. Suitable emulsifiers can include any of a wide variety of nonionic, cationic, anionic, and zwitterionic emulsifiers disclosed in the prior patents and other references. See McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973; these four references are incorporated herein by reference in their entirety.

Suitable emulsifier types include esters of glycerin, esters of propylene glycol, fatty acid esters of polyethylene glycol, fatty acid esters of polypropylene glycol (excluding the C₁-C₆ carboxylic acid esters of polypropylene glycol useful as the solid oil herein), esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps and mixtures thereof.

Suitable emulsifiers can include, but are not limited to, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, and mixtures thereof.

The emulsifiers can be used individually or as a mixture of two or more and can be included at concentrations ranging from about 0.1% to about 10%, more preferably from about 1% to about 7%, and most preferably from about 1% to about 5%, by weight of the composition.

Carboxylic Acid Copolymer Thickeners

Another component useful in the compositions herein is a carboxylic acid copolymer thickener. These crosslinked polymers contain one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. The preferred polymers for use herein are of two general types. The first type of polymer is a crosslinked homopolymer of an acrylic acid monomer or derivative thereof (e.g., wherein the acrylic acid has substituents on the two and three carbon positions independently selected from the group consisting of C₁₋₄ alkyl, —CN, —COOH, and mixtures thereof). The second type of polymer is a crosslinked copolymer having a first monomer selected from the group consisting of an acrylic acid monomer or derivative thereof (as just described in the previous sentence), a short chain alcohol (i.e. a C₁₋₄) acrylate ester monomer or derivative thereof (e.g., wherein the acrylic acid portion of the ester has substituents on the two and three carbon positions independently selected from the group consisting of C alkyl, —CN, —COOH, and mixtures thereof), and mixtures thereof; and a second monomer which is a long chain alcohol (i.e. C₈₋₄₀) acrylate ester monomer or derivative thereof (e.g., wherein the acrylic acid portion of the ester has substituents on the two and three carbon positions independently selected from the group consisting of C₁₋₄ alkyl, —CN, —COOH, and mixtures thereof). Combinations of these two types of polymers are also useful herein.

In the first type of crosslinked homopolymers the monomers are preferably selected from the group consisting of acrylic acid, methacrylic acid, ethacrylic acid, and mixtures thereof, with acrylic acid being most preferred. In the second type of crosslinked copolymers the acrylic acid monomer or derivative thereof is preferably selected from the group consisting of acrylic acid, methacrylic acid, ethacrylic acid, and mixtures thereof, with acrylic acid, methacrylic acid, and mixtures thereof being most preferred. The short chain alcohol acrylate ester monomer or derivative thereof is preferably selected from the group consisting of C₁₋₄ alcohol acrylate esters, C₁₋₄ alcohol methacrylate esters, C₁₋₄ alcohol ethacrylate esters, and mixtures thereof, with the C₁₋₄ alcohol acrylate esters, C₁₋₄ alcohol methacrylate esters, and mixtures thereof, being most preferred. The long chain alcohol acrylate ester monomer is selected from C₈₋₄₀ alkyl acrylate esters, with C₁₀₋₃₀ alkyl acrylate esters being preferred.

The crosslinking agent in both of these types of polymers is a polyalkenyl polyether of a polyhydric alcohol containing more than one alkenyl ether group per molecule, wherein the parent polyhydric alcohol contains at least 3 carbon atoms and at least 3 hydroxyl groups. Preferred crosslinkers are those selected from the group consisting of allyl ethers of sucrose and allyl ethers of pentaerythritol, and mixtures thereof. These polymers useful in the present invention are more fully described in U.S. Pat. No. 5,087,445, to Haffey et al., issued Feb. 11, 1992; U.S. Pat. No. 4,509,949, to Huang et al., issued Apr. 5, 1985; U.S. Pat. No. 2,798,053, to Brown, issued Jul. 2, 1957; which are incorporated by reference herein. See also, CTFA International Cosmetic Ingredient Dictionary, fourth edition, 1991, pp. 12 and 80; which is also incorporated herein by reference.

Examples of commercially available hompolymers of the first type useful herein include the carbomers, which are homopolymers of acrylic acid crosslinked with allyl ethers of sucrose or pentaerytritol. The carbomers are available as the Carbopol® 900 series from B.F. Goodrich. Examples of commercially available copolymers of the second type useful herein include copolymers of C₁₀₋₃₀ alkyl acrylates with one or more monomers of acrylic acid, methacrylic acid, or one of their short chain (i.e. C₁₋₄ alcohol) esters, wherein the crosslinking agent is an allyl ether of sucrose or pentaerytritol. These copolymers are known as acrylates/C10-30 alkyl acrylate crosspolymers and are commercially available as Carbopol® 1342, Pemulen TR-1, and Pemulen TR-2, from B.F. Goodrich. In other words, examples of carboxylic acid polymer thickeners useful herein are those selected from the group consisting of carbomers, acrylates/C10-C30 alkyl acrylate crosspolymers, and mixtures thereof.

The compositions of the present invention can comprise from about 0.025% to about 1%, more preferably from about 0.05% to about 0.75%, and most preferably from about 0.10% to about 0.50%, by weight of the carboxylic acid polymer thickeners.

Other Additional Components

The compositions of the present invention can comprise a wide range of other additional components. The CTFA Cosmetic Ingredient Handbook Second Edition, 1992, which is incorporated by reference herein in its entirety, describes a wide variety of nonlimiting cosmetic and pharmaceutical ingredients commonly used in hair and skin care industries, which are suitable for use in the compositions of the present invention. Nonlimiting examples of functional classes of ingredients are described at page 537 of this reference. Examples of these functional classes include: absorbents, abrasives, anti-acne agents, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, film formers, fragrance components, opacifying agents, pH adjusters, plasticizers, preservatives, propellants, reducing agents, additional skin-conditioning agents, suspending agents (nonsurfactant), ultraviolet light absorbers, and viscosity increasing agents (aqueous and nonaqueous). Examples of other functional classes of materials useful herein that are well known to one of ordinary skill in the art include solubilizing agents, sequestrants, and the like.

Nonlimiting examples of these additional components cited in the CTFA Cosmetic Ingredient Handbook, as well as other materials useful herein, include the following: vitamins and derivatives thereof [e.g., vitamin C, Vitamin A (i.e. retinoic acid), retinol, retinoids, and the like]; anti-oxidants; polyethyleneglycols; polymers for aiding the film-forming properties and substantivity of the composition (such as a copolymer of eicosene and vinyl pyrrolidone, an example of which is available from GAF Chemical Corporation as Ganex® V-220); preservatives for maintaining the antimicrobial integrity of the compositions; antioxidants; chelators and sequestrants; crosslinked and noncrosslinked nonionic and cationic polyacrylamides [e.g., Salcare SC92 which has the CTFA designation polyquaternium 32 (and) mineral oil, and Salcare SC 95 which has the CTFA designation polyquaternium 37 (and) mineral oil (and) PPG-1 trideceth-6, and the nonionic Seppi-Gel polyacrylamides available from Seppic Corp.]; and aesthetic components such as fragrances, pigments, colorings, essential oils, skin senates, astringents, skin soothing agents, skin healing agents and the like, nonlimiting examples of these aesthetic components include clove oil, menthol, camphor, eucalyptus oil, eugenol, menthyl lactate, witch hazel distillate, bisabolol, dipotassium glycyrrhizinate, and the like.

### Method of Use

The compositions of the present invention are used in conventional ways to provide cosmetic or pharmaceutical benefits appropriate to the product such as sun protection, anti-acne benefits, anti-wrinkle and anti-skin aging benefits, artificial tanning, analgesic benefits, skin conditioning benefits, facial moisturization, lip protection, skin cleansing benefits, grooming aids, and the like. Such methods of use depend upon the type of composition employed but generally involve topical application of an effective amount of the product to the hair or skin. By “effective amount” is meant an amount sufficient to provide the benefit desired. Typical amounts of the compositions of the present invention which are applied to the hair or skin will vary depending upon the type of composition and the benefit desired, however, typical ranges are generally from about 1 gram to about 25 gram, with about 2 gram being typical.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Ingredients are identified by chemical or CTFA name.

### Example 1

A topical composition in the form of an emollient cleanser is prepared by combining the following ingredients using conventional mixing techniques. This composition utilizes a mixture of a liquid polyol fatty acid polyester (liquid sucrose polyester) and a solid oil to provide emolliency and aesthetic benefits without leaving the skin feeling heavy, sticky or greasy.

| Ingredients | Weight Percent |
|---|---|
| Phase A | |
| Disodium EDTA | 0.100 |
| Glycerin | 4.000 |
| Methylparaben | 0.200 |
| Acrylates/C10-30 alkyl | 0.150 |
| acrylate Crosspolymer¹ | |
| Carbomer 954² | 0.250 |
| Water | QS 100 |
| Phase B | |
| Stearic Acid | 0.110 |
| Stearyl Alcohol | 0.875 |
| Cetyl Alcohol | 0.875 |
| Propylparaben | 0.150 |
| Steareth-2 | 0.250 |
| Steareth-21 | 0.500 |
| Liquid Sucrose Polyester³ | 2.780 |
| Ethylene Glycol Distearate | 0.220 |
| Phase C | |
| Sodium Hydroxide⁴ | 0.130 |
| Phase D | |
| Diisopropyl sebacate | 1.500 |
| Isohexadecane | 5.000 |
| Phase E | |
| Phenoxyethanol | 0.500 |
| Fragrance | 0.150 |
| Phase F | |
| Glucose Amide | 0.960 |

¹Available as Pemulen ® TR-1 from B. F. Goodrich Corporation.
²Available as Carbomer ® 954 from B. F. Goodrich Corporation.
³Liquid sucrose polyester is a mixture of hexa-, hepta-, and octa-sucrose esters, predominately the octa-ester esterified with mixed soybean oil fatty acids.
⁴50% aqueous solution.

In a suitable vessel, the Phase A ingredients are mixed at room temperature to form a dispersion and heated with stirring to 70-80° C. In a separate vessel, the Phase B ingredients are heated with stirring to 70-80° C. Phase B is then added to Phase A with mixing to form the emulsion. Next, Phase C is added to neutralize the composition. The Phase D ingredients are added with mixing, followed by cooling to 45-50° C. The Phase E ingredients are then added with stirring, followed by cooling to 40° C. Phase F is heated with mixing to 40° C. and added to the emulsion, which is cooled to room temperature.

### Example 2

A topical composition in the form of a shower product is prepared by combining the following ingredients using conventional mixing techniques. This composition utilizes a mixture of a liquid polyol fatty acid polyester (liquid sucrose polyester) and a solid oil to provide emolliency and aesthetic benefits without leaving the skin feeling heavy, sticky or greasy.

| Ingredients | Weight Percent |
|---|---|
| Sodium C12/14 Alkyl Ether | 12.0 |
| Glycerol Sulfonate | |
| Ammonium Laureth-3 | 3.00 |
| Sulfate | |
| Myristic Acid | 1.00 |
| Myristic Alcohol | 1.00 |
| Cocamidopropyl Betaine | 3.00 |
| Liquid Sucrose Polyester¹ | 15.3 |
| Petrolatum | 2.00 |
| Tetrasodium EDTA | 0.13 |
| Glycerin | 6.24 |
| Perfume | 0.80 |
| Polyquat-10 (JR-30M) | 0.30 |
| Glydant | 0.20 |
| Water | QS 100 |

¹Liquid sucrose polyester is a mixture of hexa-, hepta-, and octa-sucrose esters, predominately the octa-ester esterified with mixed soybean oil fatty acids.

In a suitable vessel, the Polyquat-10 is added to distilled water and allowed to mix until fully hydrated. The surfactants, and water soluble ingredients, are added and the mixture is heated with stirring to 70-80° C. In a separate vessel, the solid sucrose polyester is combined with the liquid oils, heated with mixing until uniform, and then added to the mixture heated at 70-80° C. The mixture is allowed to cool to 25-35° C. while continuing to stir. The glydant and perfume ingredients are then added with stirring, and the mixture is cooled to room temperature.

### Example 3

A topical composition in the form of a shower product is prepared by combining the following ingredients using conventional mixing techniques. This composition utilizes a mixture of a liquid polyol fatty acid polyester (liquid sucrose polyester) and a solid oil to provide emolliency and aesthetic benefits without leaving the skin feeling heavy, sticky or greasy.

| Ingredients | Weight Percent |
|---|---|
| Ammonium Lauryl Sulfate | 3.15 |
| Ammonium Laureth-3 | 9.45 |
| Sulfate | |
| Na Lauroamphoacetate | 5.40 |
| Polyquaternium-10 | 0.30 |
| Liquid Sucrose Polyester¹ | 15.3 |
| Behenyl behenate | 2.00 |
| Tetrasodium EDTA | 0.13 |
| Glycerin | 3.00 |
| Perfume | 0.80 |
| Citric Acid | 0.76 |
| Lauryl Alcohol | 2.00 |
| Water | QS 100 |

¹Liquid sucrose polyester is a mixture of hexa-, hepta-, and octa-sucrose esters, predominately the octa-ester esterified with mixed soybean oil fatty acids.

In a suitable vessel, the Polyquaternium-10 is added to distilled water and allowed to mix until fully hydrated. The surfactants, and water soluble ingredients, are added and the mixture is heated with stirring to 70-80° C. In a separate vessel, the solid sucrose polyester is combined with the liquid oils and lauryl alcohol, heated with mixing until uniform, and then added to the mixture heated at 70-80° C. The mixture is allowed to cool to 25-35° C. while continuing to stir. The glydant and perfume ingredients are then added with stirring, and the mixture is cooled to room temperature.

### Example 4

A topical composition in the form of a moisturizer is prepared by combining the following ingredients using conventional mixing techniques. This composition utilizes a mixture of a liquid polyol fatty acid polyester (liquid sucrose polyester) and a solid oil to provide emolliency and aesthetic benefits without leaving the skin feeling heavy, sticky or greasy.

| Ingredients | Weight Percent |
|---|---|
| Cetyl Alcohol | 1.80 |
| Stearic Acid | 0.25 |
| Stearyl Alcohol | 1.20 |
| Peg 100-stearate | 0.25 |
| Mineral Oil | 2.00 |
| Petrolatum | 1.50 |
| Isopropyl Palmitate | 1.00 |
| Cetyl Ricinoleate | 1.00 |
| Liquid Sucrose Polyester¹ | 4.00 |
| Dimethicone 350² | 0.50 |
| Propyl Paraben | 0.10 |
| Arlatone (RTM) 2l21³ | 1.00 |
| Glycerin | 9.00 |
| Urea | 2.00 |
| Octyl Methoxycinnamate | 2.00 |
| Phenoxyethanol | 0.25 |
| Carbomer 1382⁴ | 0.05 |
| Carbomer 954⁵ | 0.35 |
| Tetrasodium EDTA | 0.10 |
| Titanium Dioxide | 0.15 |
| Methyl Paraben | 0.20 |
| NaOH | 0.22 |
| Dimethicone Q-21403⁶ | 1.00 |
| Ethylene Glycol Distearate | 1.00 |
| Water | qs 100 |

¹Liquid sucrose polyester is a mixture of hexa-, hepta-, and octa-sucrose esters, predominately the octa-ester esterified with mixed soybean oil fatty acids.
²Dow Corning ® 200 Fluid (350 centistoke) from Dow Corning.
³95% by weight sorbitan stearate and 5% by weight sucrose cocoate.
⁴Carbopol ® 1382 from B. F. Goodrich.
⁵Carbopol ® 954 from B. F. Goodrich.
⁶Dow Corning ® Q-2 1403 from Dow Corning which is a mixture of 85% by weight dimethicone and 15% by weight dimethiconal.

A first premix of the liquid sucrose polyester, Arlatone 2121 and other water soluble ingredients is prepared by admixing in water and heating. A second premix of oil phase ingredients other than the silicones is prepared by mixing and heating and is added to the aqueous premix. The resulting mixture is cooled. The silicones are then added to the resulting oil-in-water emulsion and the mixture is cooled before adding minor ingredients.

What is claimed is:

1. A composition comprising a combination of a liquid polyol fatty acid polyester having a melting point of from about −30° C. to about 30° C., and a solid oil other than a solid polyol fatty acid polyester having a melting point of above about 30° C. to about 250° C., wherein said liquid polyol fatty acid polyester has a polyol moiety and at least 1 fatty acid moiety, the polyol moiety having at least 4 free hydroxyl groups wherein at least 60% of these free hydroxyl groups are esterified with one or more fatty acid moieties having from about 8 to about 22 carbon atoms.

2. The composition of claim 1 wherein the fatty acid moiety contains from about 8 to about 18 carbon atoms.

3. The composition of claim 1 wherein the polyol moiety is selected from the group consisting of erythritol, xylitol, sorbitol, glucose, sucrose, and mixtures thereof.

4. The composition of claim 3 wherein the polyol moiety is sucrose.

5. The composition of claim 4 wherein the liquid polyol fatty acid polyester has a melting point of from about −30° C. to about 25° C.

6. The composition of claim 1 wherein the solid oil has a melting point of from above about 30° C. to about 80° C.

7. The composition of claim 6 wherein the solid oil is selected from the group consisting of petrolatum, highly branched hydrocarbons, fatty alcohols, fatty acid esters, vegetable oils, hydrogenated vegetable oils, polypropylene glycols, alpha-hydroxy fatty acids, fatty acids having from about 10 to about 40 carbon atoms, alkyl amides of di-basic carboxylic acids, alkyl amides of tri-basic carboxylic acids, n-acyl amino acid derivatives, and mixtures thereof.

8. A topical composition comprising:
(a) from about 0.1% to about 99.9% by weight of a combination of a liquid polyol fatty acid polyester having a melting point of from about −30° C. to about 30° C., and a solid oil other than a solid polyol fatty acid polyester having a melting point of above about 30° C. to about 250° C., wherein said liquid polyol fatty acid polyester has a polyol moiety and at least 1 fatty acid moiety, the polyol moiety having at least 4 free hydroxyl groups wherein at least 60% of these free hydroxyl groups are esterified with one or more fatty acid moieties having from about 8 to about 22 carbon atoms; and
(b) from about 0.1% to about 99.9% by weight of a topical carrier.

9. The composition of claim 8 wherein the fatty acid moiety contains from about 8 to about 18 carbon atoms.

10. The composition of claim 8 wherein the polyol moiety is selected from the group consisting of erythritol, xylitol, sorbitol, glucose, sucrose, and mixtures thereof.

11. The composition of claim 10 wherein the polyol moiety is sucrose.

12. The composition of claim 11 wherein the liquid polyol fatty acid polyester has a melting point of from about −30° C. to about 25° C.

13. The composition of claim 8 wherein the solid oil has a melting point of from above about 30° C. to about 80° C.

14. The composition of claim 13 wherein the solid oil is selected from the group consisting of petrolatum, highly branched hydrocarbons, fatty alcohols, fatty acid esters, vegetable oils, hydrogenated vegetable oils, polypropylene glycols, alpha-hydroxy fatty acids, fatty acids having from about 10 to about 40 carbon atoms, alkyl amides of di-basic carboxylic acids, alkyl amides of tri-basic carboxylic acids, n-acyl amino acid derivatives, and mixtures thereof.

15. The composition of claim 8 wherein the topical carrier is an oil-in-water emulsion.

16. The composition of claim 8 wherein the topical carrier is an anhydous liquid solvent.

17. The composition of claim 8 wherein the composition is in the form of a hand lotion, body lotion, skin conditioning cream, skin protectant, sunscreen, cold cream, anti-acne composition, skin renewal product, non-lathering cleansing lotion, moisturizer, facial moisturizer, make-up, foundation, lipstick, lip protectant, hand cleanser, facial cleanser, body cleanser, shower product, shampoo, and mixtures thereof.

18. A method of treating human hair or skin comprising topically applying to a human in need of treatment a safe and effective amount of a composition according to claim 1.

19. A method of treating human hair or skin comprising topically applying to a human in need of treatment a safe and effective amount of a composition according to claim 8.
